(19) United States
(12) Patent Application Publication
(10) Pub. No.: US 2024/0180920 A1
(43) Pub. Date: Jun. 6, 2024

(54) CERTAIN FASCIN BINDING COMPOUNDS FOR SPINOGENESIS

(71) Applicant: Spinogenix, Inc., La Jolla, CA (US)

(72) Inventors: Stella T. Sarraf, Beverly Hills, CA (US); Vincent F. Simmon, San Diego, CA (US); Peter W. Vanderklish, San Diego, CA (US)

(21) Appl. No.: 18/552,019

(22) PCT Filed: Mar. 23, 2022
(86) PCT No.: PCT/US2022/021507

§ 371 Date: Sep. 22, 2023

Related U.S. Application Data
(60) Provisional application No. 63/291,077, filed on Dec. 17, 2021.
(60) Provisional application No. 63/165,079, filed on Mar. 23, 2021.

(51) Int. Cl. A61K 31/55; A61K 31/155; A61K 31/506; A61P 25/28

(52) U.S. Cl. CPC A61K 31/55; A61K 31/155; A61K 31/506; A61P 25/28

(57) ABSTRACT

In some embodiments, a method of promoting spinogenesis in a patient is provided, comprising administering to a patient in need thereof a therapeutically effective amount of imipramine, semapimod, or brilacidin, or a pharmaceutically acceptable salt of each thereof.

### CROSS-REFERENCE TO RELATED APPLICATIONS

This application claims the benefit under 35 U.S.C. § 119(e) of U.S. Provisional Application No. 63/165,079, filed Mar. 23, 2021, and U.S. Provisional Application No. 63/291,077, filed Dec. 17, 2021, each of which is hereby incorporated by reference in its entirety.

### FIELD

Provided herein are methods for promoting spinogenesis and for treating a neuronal disease or disorder.

### BACKGROUND

Neurological disorders are diseases of the brain, spinal cord and peripheral nervous system. The greatest societal costs, in terms of epidemiology and individual morbidity, are imposed by neurodegenerative conditions, which result in the damage or loss of neurons and the synaptic connections between them. Among the most prominent of these are Alzheimer's disease and Parkinson's disease. Other neurodegenerative conditions and age-related conditions include e.g. Parkinson's dementia, vascular dementia, Amyotrophic lateral sclerosis, frontal temporal dementia, genetic syndromes (e.g. Down syndrome), injury-related conditions (e.g. Traumatic Brain Injury, Chronic Traumatic Encephalopathy, stroke), and conditions typically considered as being purely psychiatric in nature, such as schizophrenia and depression.

Researchers have classified hundreds of diseases of the nervous system, such as brain tumors, epilepsy, Alzheimer's disease, Parkinson's disease and stroke, as well as conditions associated with old age, such as dementia. Some such conditions result from a progressive loss of synapses (junctions between two different neurons) and ultimately a loss of neurons (neurodegeneration). Unfortunately, therapeutics to effectively treat neurodegenerative diseases have been nearly impossible to develop. Neurons in the brain communicate with each other by releasing neurotransmitters (chemicals that bind to receptors on the dendrite) into the synapse, altering the electrical potential of the receiving neuron. The part of a neuron that releases the neurotransmitter is the axon (the presynaptic side of a synapse) and the part of the synapse that is affected by neurotransmitter is called a dendritic spine (the postsynaptic side of a synapse). Changes in the number, location and even shape of synaptic junctions underlie memory, learning, thinking and our personality. Various parts of the brain may be affected by neuron degeneration and suffer from a notable loss of synapses and neurons. The development of novel methods to restore spine density and replace lost synapses in the brain could have important implications for treatment of a host of neurodegenerative and developmental cognitive disorders.

Dendritic complexity, synaptogenesis, and proper development and function of neurons are endogenously regulated by growth factors such as brain derived neurotrophic factor (BDNF). While some small molecules have recently been reported to exhibit neurotrophic-like activity, these molecules have not been demonstrated to promote dendritic spine formation. The identification of a new cellular target for small molecules could lead to treatments for many neurodegenerative and mental development disorders, and would have the potential for providing improved memory and learning. Thus, small molecules that promote spine formation have potential use in ameliorating cognitive deficiencies in neurodegenerative diseases such as Alzheimer's disease, and might also find use as general cognitive enhancers. However, there is a need for pharmaceutically acceptable compounds having such activity.

### SUMMARY

Provided herein are methods for promoting spinogenesis and for treating a neuronal disease or disorder.

In some embodiments, a method of promoting spinogenesis or treating a neuronal disease or disorder in a patient is provided, comprising contacting fascin with certain compounds that interact with or inhibit the activity of fascin. In some embodiments, a method of promoting spinogenesis in a patient is provided, comprising administering to a patient in need thereof a therapeutically effective amount of imipramine, having the structure

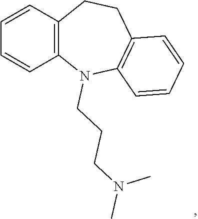 [SMILES: CN(C)CCCN1c2ccccc2CCc2ccccc21]

or a pharmaceutically acceptable salt thereof.

In some embodiments, a method of promoting spinogenesis in a patient is provided, comprising administering to a patient in need thereof a therapeutically effective amount of semapimod, having the structure

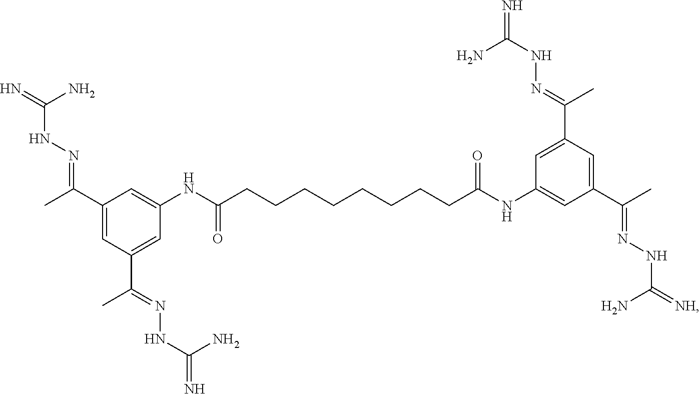 [SMILES: C/C(=N\NC(=N)N)c1cc(NC(=O)CCCCCCCCC(=O)Nc2cc(/C(C)=N/NC(=N)N)cc(/C(C)=N/NC(=N)N)c2)cc(/C(C)=N/NC(=N)N)c1]

or a pharmaceutically acceptable salt thereof, or brilacidin, having the structure

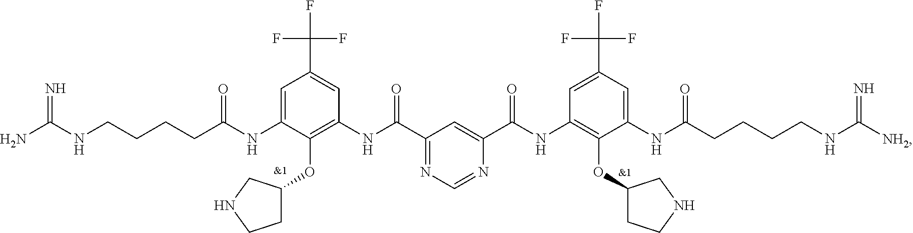 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3O[C@@H]3CCNC3)ncn2)c1O[C@@H]1CCNC1]

or a pharmaceutically acceptable salt thereof.

In some embodiments, a method of promoting spinogenesis in a neuron is provided, comprising contacting with the neuron semapimod or a pharmaceutically acceptable salt thereof, or brilacidin or a pharmaceutically acceptable salt thereof.

In some embodiments, a method of treating Alzheimer's disease is provided, comprising administering to a patient in need thereof a therapeutically effective amount of imipramine, or a pharmaceutically acceptable salt thereof.

In some embodiments, a method of treating or preventing a neuronal disease or disorder is provided, comprising administering to a patient in need thereof a therapeutically effective amount of a compound described herein or a pharmaceutically acceptable salt thereof.

In some embodiments, the neuronal disease or disorder is selected from Alzheimer's disease, Parkinson's disease, amyotrophic lateral sclerosis (ALS), frontotemporal dementia (FTD), and traumatic brain injury. In some embodiments, the neuronal disease or disorder is selected from Alzheimer's disease, Parkinson's disease, amyotrophic lateral sclerosis, frontotemporal dementia, depression and traumatic brain injury. In some embodiments, the compound is imipramine and the neuronal disease or disorder is selected from Alzheimer's disease, Parkinson's disease, amyotrophic lateral sclerosis, frontotemporal dementia, and traumatic brain injury.

In some embodiments, provided herein is a method of promoting spinogenesis in a patient, comprising administering to a patient in need thereof a therapeutically effective amount of imipramine or a pharmaceutically acceptable salt thereof.

In some embodiments, a compound that interacts with or inhibits fascin as described herein is provided, wherein the compound does not bind to fascin at binding site 1.

### BRIEF DESCRIPTION OF THE DRAWINGS

FIG. 1 is an analysis of synaptic densities in neurons following treatments with compounds according to embodiments of the present disclosure.

### DETAILED DESCRIPTION

Generally the compositions and methods described herein provide for the administration of certain compounds that interact with fascin. The compound may be imipramine, or a pharmaceutically acceptable salt thereof. In some embodiments, the compositions and methods are useful for treating a neuronal disease or disorder. The compound may be formulated in a pharmaceutical composition. A method is also provided comprising administering to a subject having a disease or condition benefitted by spinogenesis imipramine or a pharmaceutically acceptable salt thereof to a patient in need thereof. The compound may also include one or more additional pharmaceutically active materials.

The compound may be semapimod, or a pharmaceutically acceptable salt thereof. The compound may also be brilacidin, or a pharmaceutically acceptable salt thereof. In some embodiments, the compositions and methods are useful for treating a neuronal disease or disorder. The compound may be formulated in a pharmaceutical composition. A method is also provided comprising administering to a subject having a disease or condition benefitted by spinogenesis, semapimod, or a pharmaceutically acceptable salt thereof. A method is further provided comprising administering to a subject having a disease or condition benefitted by spinogenesis, brilacidin, or a pharmaceutically acceptable salt thereof. The compound may also include one or more additional pharmaceutically active materials.

I. Definitions

The following description sets forth exemplary embodiments of the present technology. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

The term “fascin” refers to a 54-58 kDa protein that is an actin cross-linking protein. The term “fascin” may refer to the amino acid sequence of human fascin 1. The term “fascin” includes both the wild-type form of the nucleotide sequences or proteins as well as any mutants thereof. In some embodiments, “fascin” is wild-type fascin. In some embodiments, “fascin” is one or more mutant forms. In some embodiments, a fascin is human fascin 1. In some embodiments, the fascin protein is encoded in the nucleotide sequence corresponding to reference number GI:347360903. In some embodiments, the fascin protein is encoded in the nucleotide sequence of RefSeq M_003088. In some embodiments, the fascin corresponds to the amino acid sequence of RefSeq NP_003079.1.

The term “spinogenesis” and the like refer, in the usual and customary sense, to development (e.g. growth and/or maturation) of dendritic spines in neurons. In some embodiments, the compounds provided herein promote spinogenesis without affecting the overall spine morphology ratio (e.g., thin, stubby, mushroom). The promotion is relative to the absence of administration of the compound.

The term “mood disorder” refers to a principally psychiatric disorder in which a patient's general emotional state or mood is distorted or inconsistent with the circumstances, and interferes with the patient's ability to carry out functions of daily life. The subject may be sad, empty or irritable, or may have periods of negative feeling alternating with feelings of excessive happiness (mania).

As used herein, the term “dendrite” refers to the branched extension of a neuron cell. Dendrites are typically responsible for receiving electrochemical signals transmitted from the axon of an adjacent neuron. The terms “dendritic spines” or “dendrite spines” refer to protoplasmic protuberances on a neuron cell (e.g., on a dendrite). In some embodiments, dendritic spines may be described as having a membranous neck which may be terminated with a capitulum (e.g., head). Dendritic spines are classified according to their shape: thin, stubby, or mushroom. Dendritic spine density refers to the total number of dendritic spines per unit length of a neuron cell. For example, the dendritic spine density may be given as the number of dendritic spines per micron.

The term “dendritic spine formation” and the like refer, in the usual and customary sense to processes which lead to an increased number of dendritic spines or increased development of dendritic spines. The term “dendritic spine morphology” and the like refer, in the usual and customary sense, to physical characterization of a dendritic spine (e.g., shape and structure). Improvement of dendritic spine morphology is a change in morphology (e.g., increase in length or increase in width) that results in increased functionality (e.g., increased number of contacts between neurons, or increased synaptic width). As known in the art and disclosed herein, exemplary methods for such characterization include measurement of the dimensions (i.e., length and width) of dendritic spines. Accordingly, the term “improving dendritic spine morphology” generally refers to an increase in length, width, or both length and width of a dendritic spine.

“Binding” refers to at least two distinct species (e.g. chemical compounds including biomolecules, or cells) to becoming sufficiently proximal to react or interact thereby resulting in the formation of a complex. For example, the binding of two distinct species (e.g., a protein and a compound described herein) may result in the formation of a complex wherein the species are interacting via non-covalent or covalent bonds. In some embodiments, the resulting complex is formed when two distinct species (e.g., a protein and a compound described herein) interact via non-covalent bonds (e.g., electrostatic, van der Waals, or hydrophobic).

As defined herein, the term “activation,” “activate,” “activating” and the like in reference to a protein-activator (e.g. agonist) interaction means positively affecting (e.g. increasing) the activity or function of the protein relative to the activity or function of the protein in the absence of the activator (e.g. compound described herein).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

“Contacting” is used in accordance with its plain ordinary meaning and refers to the process of allowing at least two distinct species (e.g. chemical compounds including biomolecules, or cells) to become sufficiently proximal to interact. The term “contacting” may include allowing two molecular species to react or physically touch, wherein the two species may be, for example, a compound as described herein, a biomolecule, a protein or an enzyme. In some embodiments contacting includes allowing a compound described herein to interact with a protein (e.g., fascin) or enzyme. In some embodiments, contacting may comprise binding a protein.

As defined herein, the terms “inhibition,” “inhibit,” “inhibiting” and the like, are to be given their customary meanings to those of skill in the art. In reference to a protein-inhibitor (e.g. antagonist) interaction, the terms “inhibition,” “inhibit,” “inhibiting” mean negatively affecting (e.g. decreasing) the functional activity of the protein relative to the functional activity of the protein in the absence of the inhibitor.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named.

The prefix “Cᵤ₋ᵥ” indicates that the following group has from u to v carbon atoms. For example, “C₁₋₆ alkyl” indicates that the alkyl group has from 1 to 6 carbon atoms.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

Some compounds exist as tautomers. Tautomers are in equilibrium with one another. For example, amide containing compounds may exist in equilibrium with imidic acid tautomers. Regardless of which tautomer is shown, and regardless of the nature of the equilibrium among tautomers, the compounds are understood by one of ordinary skill in the art to comprise all tautomers.

Any formula or structure given herein, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure, or counter-ions thereto, include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P, ³²p, ³⁵S, ³⁶Cl and ¹²⁵I. Various isotopically labeled compounds are possible under the present disclosure, for example those into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The disclosure also includes “deuterated analogs” of compounds, and counter-ions thereto, in which from 1 to n hydrogens attached to a carbon atom is/are replaced by deuterium, in which n is the number of hydrogens in the molecule. Such compounds exhibit increased resistance to metabolism and are thus useful for increasing the half-life of a compound when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in a compound.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition. Accordingly, in the compounds of this disclosure any atom specifically designated as a deuterium (D) is meant to represent deuterium.

Compounds described herein may be present as a salt, such as a pharmaceutically acceptable salt. Compounds are capable of forming salts such as acid and/or base salts. Provided are also pharmaceutically acceptable salts, hydrates, solvates, tautomeric forms, polymorphs, and prodrugs of the compounds described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use. Salts of compounds described herein can be prepared according to procedures described herein and as known in the art.

The term “pharmaceutically acceptable salt” of a given compound, refers to salts that retain the biological effectiveness and properties of the given compound, and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₂), tri(substituted alkyl) amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂(substituted alkenyl)), di(substituted alkenyl) amines (i.e., HN(substituted alkenyl)₂), tri(substituted alkenyl) amines (i.e., N(substituted alkenyl)₃, mono-, di- or tri-cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃), or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like. Methods of preparing a salt also include mixing a compound by redox reaction with an active metal, or by exchange of ions, for example, due to differing solubility of salts.

A “solvate” is a solid form of a compound in which solvent molecules are incorporated. A solvate is formed by the interaction of a solvent and a compound. A hydrate is a solvate in which the solvent is water. Solvates of salts of compounds described herein are also provided.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

“Treatment” or “treating” is an approach for obtaining beneficial or desired results including clinical results. Beneficial or desired clinical results may include one or more of the following: a) treating the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival).

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal. In one embodiment, the subject is a human. When the subject is a human person, the subject may be referred to as a “patient.”

The term “therapeutically effective amount” or “effective amount” of a compound described herein or a pharmaceutically acceptable salt thereof means an amount sufficient to effect treatment when administered to a subject, to provide a therapeutic benefit such as amelioration of symptoms or slowing of disease progression. For example, a therapeutically effective amount may be an amount sufficient to decrease a symptom of a neuronal disease. The therapeutically effective amount may vary depending on the subject, and disease or condition being treated, the weight and age of the subject, the severity of the disease or condition, and the manner of administering, which can readily be determined by one or ordinary skill in the art.

The methods described herein may be applied to cell populations in vivo or ex vivo. “In vivo” means within a living individual, as within an animal or human. In this context, the methods described herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. In this context, the compounds and compositions described herein may be used for a variety of purposes, including therapeutic and experimental purposes. For example, the compounds and compositions described herein may be used ex vivo to determine the optimal schedule and/or dosing of administration of a compound of the present disclosure for a given indication, cell type, individual, and other parameters. Information gleaned from such use may be used for experimental purposes or in the clinic to set protocols for in vivo treatment. Other ex vivo uses for which the compounds and compositions described herein may be suited are described below or will become apparent to those skilled in the art. The selected compounds may be further characterized to examine the safety or tolerance dosage in human or non-human subjects. Such properties may be examined using commonly known methods to those skilled in the art.

### Compounds

Provided herein are agents that promote spinogenesis. Such agents are useful in the treatment of neuronal diseases and disorders.

In some embodiments, the spinogenesis-promoting compound is imipramine, having the structure

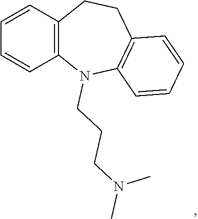 [SMILES: CN(C)CCCN1c2ccccc2CCc2ccccc21]

or a pharmaceutically acceptable salt thereof. The salt of imipramine may be imipramine hydrochloride.

In some embodiments, the spinogenesis-promoting compound is semapimod, having the structure

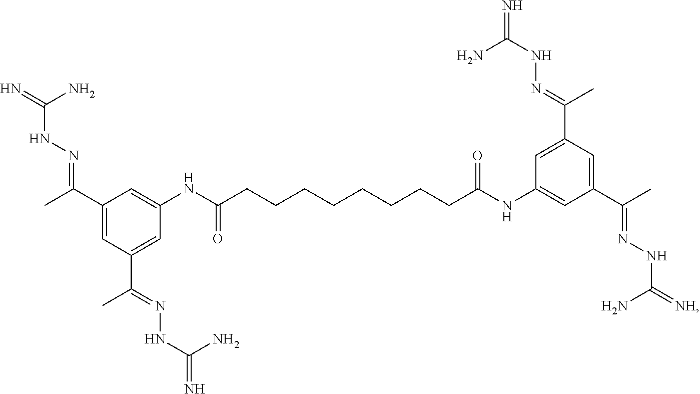 [SMILES: C/C(=N\NC(=N)N)c1cc(NC(=O)CCCCCCCCC(=O)Nc2cc(/C(C)=N/NC(=N)N)cc(/C(C)=N/NC(=N)N)c2)cc(/C(C)=N/NC(=N)N)c1]

or a pharmaceutically acceptable salt thereof. The salt of semapimod may be semapimod hydrochloride.

In some embodiments, the spinogenesis-promoting compound is brilacidin, having the structure

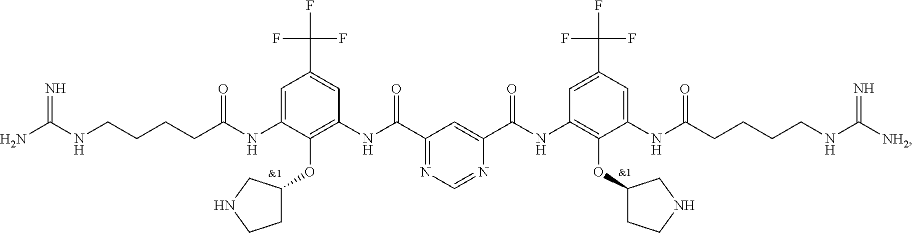 [SMILES: N=C(N)NCCCCC(=O)Nc1cc(C(F)(F)F)cc(NC(=O)c2cc(C(=O)Nc3cc(C(F)(F)F)cc(NC(=O)CCCCNC(=N)N)c3O[C@@H]3CCNC3)ncn2)c1O[C@@H]1CCNC1]

or a pharmaceutically acceptable salt thereof. The salt of brilacidin may be brilacidin hydrochloride.

Compounds described herein can be prepared according to methods known to those of skill in the art. If available, compounds may be purchased commercially, e.g., from Sigma Aldrich or other chemical suppliers.

Synthesis can be conducted by known procedures or modifications thereof. For example, many of the starting materials are available from commercial suppliers such as Aldrich Chemical Co. (Milwaukee, Wisconsin, USA), Bachem (Torrance, California, USA), Emka-Chemce or Sigma (St. Louis, Missouri, USA). Others may be prepared by procedures or modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989) organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

### Treatment Methods and Uses

### Fascin

Described herein are methods for the regeneration of spine synapses lost to neurodegenerative conditions by targeting a cytoskeletal protein with an agent such as a compound described herein. Unexpectedly, it was observed that interaction with or inhibition of the cytoskeletal protein fascin 1 (FSCN1) resulted in a rapid upregulation of dendritic spines in vivo and in vitro. Dendritic spines contain filamentous actin (F-actin), a cytoskeletal polymer that gives structure to cells and their subcellular specializations. Extension of F-actin filaments and changes in their organization are important for the formation, maturation, and plasticity of dendritic spines. Prior to the present disclosure, it was believed that the ability of fascin 1 to bundle F-actin filaments into parallel arrays would be essential for the formation of a wide range of cellular protrusions, such as invadopodia, filapodia and possibly dendritic spines. Fascin 1 is believed to promote cellular migration and the related process of cancer metastasis by such processes. Small molecule inhibitors of fascin 1 that block its ability to bundle F-actin have been observed to reduce F-actin-rich cellular protrusions. Thus, prior work suggested that fascin inhibitors would also block the formation of dendritic spines, which are cellular protrusions rich in F-actin. However, contrary to expectation the opposite is true: that structurally distinct inhibitors of fascin 1, as well as genetic knockdown of fascin 1, may result in a rapid increase in dendritic spine density. Without wishing to be limited by theory, it is believed that dendritic spines require the formation of highly branched assemblies of F-actin, and formation of such assemblies may be precluded, or significantly reduced, by bundling F-actin into parallel arrays by fascin 1.

Fascin binding sites are described in International Publication No. WO 2020/046991. In some embodiments, a method of interacting with or binding a fascin protein at site 2 or site 3 is provided, the method comprising contacting the fascin protein with an effective amount of imipramine or a pharmaceutically acceptable salt thereof. In some embodiments, the method causes interaction with fascin. In some embodiments, the method inhibits fascin. It is believed that imipramine or a pharmaceutically acceptable salt thereof, promote dendritic spine formation by interacting with or inhibiting fascin.

In some embodiments, a method of interacting with or binding a fascin protein at site 2 is provided, the method comprising contacting the fascin protein with an effective amount of imipramine, or a pharmaceutically acceptable salt thereof. In some embodiments, a method of interacting with or binding a fascin protein at site 3 is provided, the method comprising contacting the fascin protein with an effective amount of semapimod, or a pharmaceutically acceptable salt thereof. In some embodiments, a method of interacting with or binding a fascin protein at site 3 is provided, the method comprising contacting the fascin protein with an effective amount of brilacidin, or a pharmaceutically acceptable salt thereof. In some embodiments, the method causes interaction with fascin. In some embodiments, the method inhibits fascin. It is believed that imipramine, or a pharmaceutically acceptable salt thereof, semapimod, or a pharmaceutically acceptable salt thereof, brilacidin, or a pharmaceutically acceptable salt thereof, promote dendritic spine formation by interaction with or inhibiting fascin.

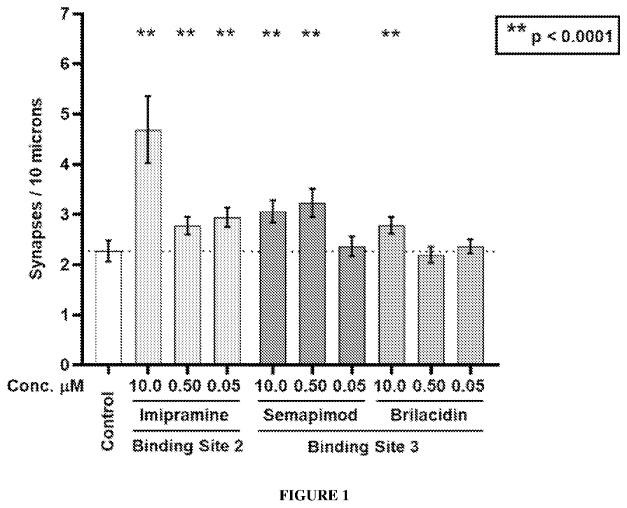

Fascin is an important actin cross-linker that has no amino-acid sequence homology with other actin-binding proteins. Three forms of fascin are found in vertebrates: fascin 1, widely found in the nervous system and elsewhere; fascin 2 found in the retinal photoreceptor cells; and fascin 3, which is only found in the testes. In some embodiments, a fascin is human fascin 1. Fascin has a molecular mass of 55 kDa, functions as a monomeric entity, and cross-links actin filaments into straight, compact and rigid bundles, to impart mechanical stiffness to actin bundles. It is believed that fascin holds parallel actin filaments together to form filopodia on the order of 60-200 nm in diameter. A structure of fascin, along with actin binding sites, is illustrated in FIG. 1 of International Patent Publication No. WO 2020/046991.

During neuron development, it is believed that long bundles of f-actin push out the membrane of the neuron to form structures such as axons, dendrites, filopodia and lamellipodia. Fascin is thought to be involved in cytoskeletal reorganization of nascent dendritic protrusions. Thus, actin bundling by fascin is generally believed to be required for the formation and extension of axons and dendrites. Surprisingly, the present results indicate that interaction with or inhibiting the activity of fascin in formation of actin bundles promotes the formation of dendritic spines, protrusions of the cytoplasmic membrane of dendrites.

Fascin is believed to have at least three actin binding sites, binding site 1, binding site 2 and binding site 3. Thus, fascin appears to have three sites at which actin may be bound. Binding site 2 was not seen in early apo (ligand-free) crystal structures of fascin, perhaps due to movement of the protein structure upon ligand binding. Compounds disclosed in International Patent Publication No. WO 2017/120198 are believed to bind fascin at binding site 1. However, it is emphasized that the mode of action is not well understood.

In some embodiments, fascin binding site 1 is defined, at least in part, by V10, Q11, L40, K41, A137, H139, Q141, Q258, S259, R383, R389, E391, G393, F394, S409, Y458, K460, E492, and/or Y493. In some embodiments, imipramine or a pharmaceutically acceptable salt thereof, does not bind to any of fascin residue selected from V10, Q11, L40, K41, A137, H139, Q141, Q258, S259, R383, R389, E391, G393, F394, S409, Y458, K460, E492, and/or Y493. In some embodiments, imipramine or a pharmaceutically acceptable salt thereof does not bind to fascin binding site 1 and/or binding site 3. In some embodiments, semapimod, or a pharmaceutically acceptable salt thereof, or brilacidin, or a pharmaceutically acceptable salt thereof, does not bind to any of fascin residue selected from V10, Q11, L40, K41, A137, H139, Q141, Q258, S259, R383, R389, E391, G393, F394, S409, Y458, K460, E492, and/or Y493. In some embodiments, semapimod, or a pharmaceutically acceptable salt thereof, or brilacidin, or a pharmaceutically acceptable salt thereof does not bind to fascin binding site 1.

Fascin binding site 2 is believed to be defined, at least in part, by F14, L16, L48, Q50, L62, W101, L103, E215, and/or S218. In some embodiments, imipramine or a pharmaceutically acceptable salt thereof, binds to at least one fascin residue selected from F14, L16, L48, Q50, L62, W101, L103, E215, and S218. In some embodiments, imipramine or a pharmaceutically acceptable salt thereof, binds to two, three, four, five, six, seven, or eight fascin residues selected from F14, L16, L48, Q50, L62, W101, L103, E215, and S218. In some embodiments, imipramine or a pharmaceutically acceptable salt thereof, binds to at least one group I fascin residue selected from F14 and L16. In some embodiments, imipramine or a pharmaceutically acceptable salt thereof, binds to at least one group II fascin residue selected from L48, Q50, and L62. In some embodiments, imipramine or a pharmaceutically acceptable salt thereof, binds to at least one group III fascin residue selected from W101 and L103. In some embodiments, imipramine or a pharmaceutically acceptable salt thereof, binds to at least one group IV fascin residue selected from E215 and S218. In some embodiments, fascin binding site 2 is defined, at least in part, by F14, L16, L48, A58, V60, L62, I93, A95, W101, L103, V134, T213, L214, E215, F216, and/or R217.

In some embodiments, semapimod, or a pharmaceutically acceptable salt thereof, or brilacidin, or a pharmaceutically acceptable salt thereof, does not bind to any of fascin residue selected from F14, L16, L48, Q50, L62, W101, L103, E215, and/or S218. In some embodiments, semapimod, or a pharmaceutically acceptable salt thereof, or brilacidin, or a pharmaceutically acceptable salt thereof, does not bind to any of fascin residue selected from F14, L16, L48, A58, V60, L62, I93, A95, W101, L103, V134, T213, L214, E215, F216, and/or R217. In some embodiments, semapimod, or a pharmaceutically acceptable salt thereof, or brilacidin, or a pharmaceutically acceptable salt thereof does not bind to fascin binding site 2.

In some embodiments, fascin binding site 3 is defined, at least in part, by Q291, R308, H310, T311, G312, K313, Y314, L317, T318, T320, T326, S328, K329, N330, N331, S333, E339, R341, R344, R348, K353, S350, N351, F354, T356, S357, K358, K359, N360, Q362, L363, S366, V367, E368, T369, D372, S373, L375, L377, I381, and/or K379. In some embodiments, imipramine or a pharmaceutically acceptable salt thereof, binds to at least one fascin residue selected from Q291, R308, H310, T311, G312, K313, Y314, L317, T318, T320, T326, S328, K329, N330, N331, S333, E339, R341, R344, R348, K353, S350, N351, F354, T356, S357, K358, K359, N360, Q362, L363, S366, V367, E368, T369, D372, S373, L375, L377, 1381, and K379. In some embodiments, imipramine or a pharmaceutically acceptable salt thereof, binds to two, three, four, five, six, seven, or eight fascin residues selected from Q291, R308, H310, T311, G312, K313, Y314, L317, T318, T320, T326, S328, K329, N330, N331, S333, E339, R341, R344, R348, K353, S350, N351, F354, T356, S357, K358, K359, N360, Q362, L363, S366, V367, E368, T369, D372, S373, L375, L377, I381, and K379.

In some embodiments, semapimod, or a pharmaceutically acceptable salt thereof, or brilacidin, or a pharmaceutically acceptable salt thereof, binds to at least one fascin residue selected from Q291, R308, H310, T311, G312, K313, Y314, L317, T318, T320, T326, S328, K329, N330, N331, S333, E339, R341, R344, R348, K353, S350, N351, F354, T356, S357, K358, K359, N360, Q362, L363, S366, V367, E368, T369, D372, S373, L375, L377, I381, and K379. In some embodiments, semapimod, or a pharmaceutically acceptable salt thereof, or brilacidin, or a pharmaceutically acceptable salt thereof, binds to two, three, four, five, six, seven, or eight fascin residues selected from Q291, R308, H310, T311, G312, K313, Y314, L317, T318, T320, T326, S328, K329, N330, N331, S333, E339, R341, R344, R348, K353, S350, N351, F354, T356, S357, K358, K359, N360, Q362, L363, S366, V367, E368, T369, D372, S373, L375, L377, I381, and K379.

In some embodiments a binding site of imipramine or a pharmaceutically acceptable salt thereof, can be determined by site-directed mutagenesis. For example, an amino acid residue in a mutant fascin may be changed relative to a wild-type fascin. For example, if a reduction in binding affinity of an agent is determined between a wild type fascin and a mutated fascin having replacement of an amino acid residue at binding site 1, binding site 2, or binding site 3 with a non-native residue, e.g., an alanine residue, then the reduction can be attributed to a loss of affinity for binding at the site of replacement. Site-directed mutagenesis may be carried out according a known method, for example, Kunkel's method, Cassette mutagenesis, PCR site-directed mutagenesis, or CRISPR.

Likewise, in some embodiments a binding site of semapimod, or a pharmaceutically acceptable salt thereof, or brilacidin, or a pharmaceutically acceptable salt thereof, can similarly be determined by site-directed mutagenesis, such as described herein including embodiments,

In some embodiments, imipramine or a pharmaceutically acceptable salt thereof, binds to fascin with a Ka of at least about 10 nM, at least about 100 nM, at least about 1 M, at least about 5 μM, at least about 10 μM, at least about 20 μM, at least about 50 μM, at least about 100 μM, or at least about 500 M as determined by isothermal titration calorimetry.

In some embodiments, semapimod, or a pharmaceutically acceptable salt thereof, or brilacidin, or a pharmaceutically acceptable salt thereof, binds to fascin with a Ka of at least about 10 nM, at least about 100 nM, at least about 1 μM, at least about 5 μM, at least about 10 μM, at least about 20 μM, at least about 50 μM, at least about 100 μM, or at least about 500 μM as determined by isothermal titration calorimetry.

In some embodiments, other compounds beyond those described above (e.g. brilacidin and semapimod) which bind at the fascin binding site 3 (“site 3-binding compounds”), or a pharmaceutically acceptable salt thereof, may be used in place of, or along with, those described herein including embodiments. Such compounds include, for example, Urea C-13 (or (13C)urea), Uracil (or 1,2,3,4-tetrahydropyrimidine-2,4-dione), N-(7-carbamimidoylnaphthalen-1-yl)-3-hydroxy-2-methylbenzamide, N-[2-({ [amino(imino)methyl]amino }oxy)ethyl]-2-{6-chloro-3-[(2,2-difluoro-2-phenylethyl)amino]-2-fluorophenyl }acetamide, 6-carbamimidoyl-4-(3-hydroxy-2-methyl-benzoylamino)-naphthalene-2-carboxylic acid methyl ester (or methyl 6-carbamimidoyl-4-(3-hydroxy-2-methylbenzamido)naphthalene-2-carboxylate), pantothenyl-aminoethanol-acetate pivalic acid, [4-(2-amino-4-methyl-thiazol-5-yl)-pyrimidin-2-yl]-(3-nitro-phenyl)-amine.

In some embodiments, other compounds which bind at the fascin binding site 1 (“site 1-binding compounds”), or a pharmaceutically acceptable salt thereof, may be used in place of, or along with, those described herein including embodiments. Such compounds include, for example, (2R)-14-fluoro-2-methyl-6,9,10,19-tetraazapentacyclo[14.2.1.0\hat{ }{2,6}.0\hat{ }{8,18}.0\hat{ }{12,17}]nonadeca-1(18),8,12(17), 13,15-pentaen-11-one, Hexoprenaline, N-(2-amino-4-fluorophenyl)-4-{[(2E)-3-(pyridin-3-yl)prop-2-enamido]methyl}benzamide, N-(4-sulfamoylphenyl)-1H-indazole-3-carboxamide, 2-hydroxy-5-(3,5,7-trihydroxy-4-oxochromen-2-yl)phenoxyphosphonic acid, Allantoin, 4-[(4E)-1-(carboxymethyl)-4-[(4-hydroxyphenyl)methylidene]-5-oxoimidazol-2-yl]-4-iminobutanoic acid, N-(1H-indazol-5-yl)-2-(6-methylpyridin-2-yl)quinazolin-4-amine, chlorogenic acid.

### Neuronal Diseases and Conditions and Treatment Thereof

A unifying feature of neurodegenerative conditions with a cognitive component is the loss of synapses that utilize the amino acid glutamate as a neurotransmitter (“glutamatergic” synapses), which in humans and other mammals are believed to be the most abundant type of synapse. Importantly, about 90% of glutamatergic synapses involve a post-synaptic dendritic spine. The majority of synapses lost in neurodegenerative conditions are those in which the axon makes contact with a dendritic spine, so-called “axospinous synapses.” Under normal conditions, changes in the density, shape, and protein composition of dendritic spines impact the strength of synaptic communication, and are the basis of several forms of synaptic change (i.e. “plasticity”) involved in learning and memory, cognitive flexibility, adaptation to injury and disease, and other processes. These changes in axospinous synapses are believed to be important for the memory encoding functions of structures such as the hippocampus. Accordingly, an early and progressive loss of dendritic spines in hippocampus and other regions is believed to be a driver of memory loss and cognitive decline in Alzheimer's disease and other dementias. The development of novel methods to regenerate spine density could have important implications for treatment of a host of neurodegenerative and developmental cognitive disorders.

Dendritic spines are specialized protrusions responsible for receiving synaptic inputs, providing an important function in communication between neurons. The morphology of dendritic spines and their overall density correlates with synaptic function and are strongly implicated in memory and learning. Cellular changes in brain cells may contribute to pathogenesis of a neuronal disease. For example, an aberrant level (e.g., reduction) in dendritic spine density in the brain may contribute to the pathogenesis of the neuronal disease. Consequently, alteration or misregulation of dendritic spines is believed to influence synaptic function and play a major role in various neurological and psychiatric disorders such as autism, fragile X syndrome, Parkinson's Disease (PD) and Alzheimer's Disease (AD). For example, in AD there is mounting evidence suggesting deficits begin with alterations of hippocampal synaptic function prior to neuronal loss, which may or may not be caused by amyloid-β (Aβ) protein. Therefore, treatment strategies that target the initial synaptic loss, rather than late stage disease intervention, or even reduction of Aβ, may provide a better prognosis for the treatment of AD. For example, Fragile X syndrome is characterized by an overabundance of immature spines.

Provided herein are methods useful for promoting spinogenesis. In some embodiments, the method comprises administering to the subject an effective amount of imipramine or a pharmaceutically acceptable salt thereof, as described herein including embodiments. Spinogenesis may be observed as an increase in the average number of spines per neuron, or a unit length of a neuron, which may be referred to as an increase in dendritic spine density. Spinogenesis may be observed as an improvement in dendritic spine morphology. For example, an improvement in dendritic spine morphology may be observed as an increase in average size of spine heads. Spinogenesis may be observed as an improvement in dendritic spine size, spine plasticity, spine motility, spine density and/or synaptic function. Spinogenesis may be observed as an increase in local spatial average of membrane potential. Spinogenesis may be observed as an increase in postsynaptic concentration (e.g., volume-averaged) of Ca²⁺. Spinogenesis may be observed as an increase in the average ratio of matured to immature spines, such as in the ration of “mushroom shaped” or “stubby” spines relative to thin spines. In some embodiments, imipramine or a pharmaceutically acceptable salt thereof, increases the dendritic spine density relative to a control. In some embodiments, imipramine, or a pharmaceutically acceptable salt thereof, increases the dendritic spine density relative to that observed at the time that treatment is initiated. In some embodiments, the increase in dendritic spine density results in a reduction in symptoms of a neuronal disease or disorder in a subject or patient. In some embodiments, the increase in dendritic spine density is accounted for by anatomical observation. In some embodiments, the increase in dendritic spine density is observed in primary hippocampal neurons.

In some embodiments, the method comprises administering to the subject an effective amount of imipramine, or a pharmaceutically acceptable salt thereof, as described herein including embodiments.

In some embodiments, the average dendritic spine density, relative to the time that treatment with imipramine or a pharmaceutically acceptable salt thereof, is initiated, increases by at least about 5%, 10%, 15%, 20%, 25%, 30%, 35%, 40%, 45%, or 50%, or any range between any two of the numbers, end points inclusive. In some embodiments, the degree of spinogenesis is proportional to the deficit in spine density present before treatment and the treatment returns spine density to levels that are considered as normal or physiologically relevant. In some embodiments, the duration of treatment with imipramine or a pharmaceutically acceptable salt thereof, is 15 minutes, 30 minutes, 1 hour, 2 hours, 4 hours, 8 hours, 1 day, 3 days, 5 days, 7 days, 14 days, 28 days, 90 days, 180 days, or 365 days. In some embodiments, the administration is not continuous, and is, for example, conducted in discrete administrations on the specified days.

In some embodiments, the method increases spine density through promoting the formation of new spines. In some embodiments, the method increases the average spine density by at least about 5%, 10%, 15%, 20%, 25%, 30%, 35%, 40%, 45%, or 50%, or any range between any two of the numbers, end points inclusive, relative to a control (e.g., the spine density in the absence of the compound). In some embodiments, the method increases the average spine density about 50% relative to a control (e.g., the spine density in the absence of the compound).

In some embodiments, the method increases the average number of spines per neuron, relative to the time that treatment with imipramine or a pharmaceutically acceptable salt thereof, is initiated. In some embodiments, average number spines per unit length of a neuron increases by at least about 10, 20, 30, 40, 50, 60 more, or any range between any two of the numbers, end points inclusive. In some embodiments, the time is 1 hour, 2 hours, 4 hours, 8 hours, 1 day, 3 days, 5 days, 7 days, 14 days, 28 days, 90 days, 180 days, or 365 days.

In some embodiments, the method useful for promoting spinogenesis comprises administering to the subject an effective amount of semapimod, or a pharmaceutically acceptable salt thereof, as described herein including embodiments. In some embodiments, the method comprises administering to the subject an effective amount of brilacidin, or a pharmaceutically acceptable salt thereof, as described herein including embodiments. In some embodiments, semapimod, or a pharmaceutically acceptable salt thereof, or brilacidin, or a pharmaceutically acceptable salt thereof, increases the dendritic spine density relative to a control. In some embodiments, semapimod, or a pharmaceutically acceptable salt thereof, or brilacidin, or a pharmaceutically acceptable salt thereof, increases the dendritic spine density relative to that observed at the time that treatment is initiated. In some embodiments, the average dendritic spine density, relative to the time that treatment with imipramine or a pharmaceutically acceptable salt thereof, is initiated, increases by at least about 5%, 10%, 15%, 20%, 25%, 30%, 35%, 40%, 45%, or 50%, or any range between any two of the numbers, end points inclusive.

In some embodiments, the degree of spinogenesis is proportional to the deficit in spine density present before treatment and the treatment returns spine density to levels that are considered as normal or physiologically relevant. In some embodiments, the dendritic spine density, relative to the time that treatment with semapimod, or a pharmaceutically acceptable salt thereof, or brilacidin, or a pharmaceutically acceptable salt thereof, is initiated, increases by about 50% to about 100%. In some embodiments, the duration of treatment with semapimod, or a pharmaceutically acceptable salt thereof, or brilacidin, or a pharmaceutically acceptable salt thereof, is 15 minutes, 30 minutes, 1 hour, 2 hours, 4 hours, 8 hours, 1 day, 3 days, 5 days, 7 days, 14 days, 28 days, 90 days, 180 days, or 365 days. In some embodiments, the administration is not continuous, and is, for example, conducted in discrete administrations on the specified days.

In some embodiments, the method increases the average number of spines per neuron, relative to the time that treatment with semapimod, or a pharmaceutically acceptable salt thereof, or brilacidin, or a pharmaceutically acceptable salt thereof, is initiated. In some embodiments, average number spines per unit length of a neuron increases by at least about 10, 20, 30, 40 more, or any range between any two of the numbers, end points inclusive. In some embodiments, the time is 1 hour, 2 hours, 4 hours, 8 hours, 1 day, 3 days, 5 days, 7 days, 14 days, 28 days, 90 days, 180 days, or 365 days.

In some embodiments, the compounds are useful in the treatment of neuronal diseases and disorders. A neuronal disease is a disease or condition in which the function of a subject's nervous system becomes impaired. The neuronal disease or disorder may be a neurological disease or disorder. The neuronal disease or disorder may be associated with a neurodegenerative disease or disorder.

In an aspect is provided a method of treating a neuronal disease in a patient in need thereof, the method comprising administering a therapeutically effective amount of imipramine or a pharmaceutically acceptable salt thereof, to the patient. In some embodiments, the neuronal disease is Alzheimer's disease. In some embodiments, the neuronal disease is amyotrophic lateral sclerosis (ALS). In some embodiments, the neuronal disease is frontotemporal dementia (FTD). In some embodiments, the neuronal disease is Parkinson's disease. In some embodiments, the neuronal disease is Parkinson's dementia. In some embodiments, the neuronal disease is autism. In some embodiments, the neuronal disease is fragile X syndrome, Angelman syndrome, or other neurodevelopmental disorders characterized by abnormally low levels of mature spines or total spine density. In some embodiments, the disease or disorder is related to (e.g. characterized by) an accumulation of amyloid plaques. In some embodiments, the neuronal disease is a traumatic brain injury. In some embodiments, a patient having a neuronal disease has suffered a traumatic brain injury before, during, or after the onset of the neuronal disease. In some embodiments, the neuronal disease includes a neuronal impairment. A neuronal impairment may include atrophy or other decrease in the effective functioning of the neuron. For example, it is known that Alzheimer's disease presents with neuronal impairment, especially in cortical neurons, e.g., hippocampal neurons and neurons in proximity to the hippocampus. Loss of synapses may correlate with loss of dendritic spines and neurodegeneration.

In some aspects, the method of treating a neuronal disease in a patient in need thereof, such as those described herein including embodiments, comprises administering a therapeutically effective amount of semapimod, or a pharmaceutically acceptable salt thereof, or brilacidin, or a pharmaceutically acceptable salt thereof, to the patient.

In some embodiments, the neuronal disease is associated with abnormal dendritic spine morphology, spine size, spine plasticity, spine motility, spine density and/or abnormal synaptic function. In some embodiments, the neuronal disease is associated with an abnormal (e.g., reduced) level of dendritic spine density.

In some embodiments, the neuronal disease is Alzheimer's disease. In some embodiments, the neuronal disease is Parkinson's disease. In some embodiments, the neuronal disease is Parkinson's disease accompanied by dementia. In some embodiments, the neuronal disease is autism. In some embodiments, the neuronal disease is stroke. In some embodiments, the neuronal disease is posttraumatic stress disorder (PTSD). In some embodiments, the neuronal disease is traumatic brain disorder (TBD). In some embodiments, the neuronal disease is chronic traumatic encephalopathy (CTE). In some embodiments, the neuronal disease is schizophrenia. In some embodiments, the neuronal disease is dementia (e.g., general dementia). In some embodiments, the neuronal disease is attention- deficit/hyperactivity disorder (ADHD). In some embodiments, the neuronal disease is amyotrophic lateral sclerosis (ALS). In some embodiments, the neuronal disease is frontotemporal lobar degeneration (FTLD) (e.g., FTLD-tau, FTLD-TDP, or FTLD-FUS), or ALS/FTD. In some embodiments, the neuronal disease is memory loss. In some embodiments, the neuronal disease includes memory loss. In some embodiments, the neuronal disease is age-related memory loss. In some embodiments, the neuronal disease includes age-related memory loss. In some embodiments, the neuronal disease is hypertensive encephalopathy. In some embodiments, the neuronal disease is chronic stress. In some embodiments, the neuronal disease includes chronic stress. In some embodiments, the neuronal disease is FTLD-TDP Type A. In some embodiments, the neuronal disease is FTLD-TDP Type B. In some embodiments, the neuronal disease is FTLD-TDP Type C. In some embodiments, the neuronal disease is FTLD-TDP Type D.

In some embodiments, the neuronal disease or disorder is amyotrophic lateral sclerosis (ALS), in which spine synapse loss in motor cortex and on spinal motor neurons contributes to movement dysfunction. In some embodiments, the neuronal disorder is a mixture of motor and cognitive/dementia disorders that involves symptoms of ALS and FTD, which are thought to represent different ends of a spectrum of genetically and mechanistically related neurodegenerative diseases. The neuronal disease or disorder may be a tauopathy.

The neuronal disease or condition may be schizophrenia. The symptoms of schizophrenia generally fall into the following three categories: Psychotic symptoms include altered perceptions (e.g., changes in vision, hearing, smell, touch, and taste), abnormal thinking, and odd behaviors. People with psychotic symptoms may lose a shared sense of reality and experience themselves and the world in a distorted way. Specifically, individuals typically experience: hallucinations, delusions, and thought disorders (such as nonconventional thinking and/or disorganized speech). Negative symptoms include reduced motivation, difficulty planning, diminished feelings of pleasure, flat affect, and reduced verbalization. Cognitive symptoms include difficulty processing information, difficulty focusing, and difficult sustaining attention.

Examples of neuronal diseases that may be treated with a compound or method described herein include Alexander's disease, Alper's disease, Alzheimer's disease, depression, perinatal asphyxia, Parkinson's disease dementia (“PD dementia”), amyotrophic lateral sclerosis, ataxia telangiectasia, Batten disease (also known as Spielmeyer-Vogt-Sjogren-Batten disease), spongiform encephalopathy (e.g., bovine spongiform encephalopathy (mad cow disease), Kuru, Creutzfeldt-Jakob disease, fatal familial insomnia, Canavan disease, Cockayne syndrome, corticobasal degeneration, fragile X syndrome, frontotemporal dementia, Gerstmann-Straussler-Scheinker syndrome, Huntington's disease, HIV-associated dementia, Kennedy's disease, Krabbe's disease,

Lewy body dementia, Machado-Joseph disease (Spinocerebellar ataxia type 3), multiple sclerosis, multiple system atrophy, narcolepsy, neuroborreliosis, Parkinson's disease, Pelizaeus-Merzbacher Disease, Pick's disease, primary lateral sclerosis, prion diseases, Refsum's disease, Sandhoff s disease, Schilder's disease, subacute combined degeneration of spinal cord secondary to pernicious anaemia, schizophrenia, spinocerebellar ataxia (multiple types with varying characteristics), spinal muscular atrophy, Steele-Richardson-Olszewski disease, Tabes dorsalis, drug-induced Parkinsonism, progressive supranuclear palsy, corticobasal degeneration, multiple system atrophy, idiopathic Parkinson's disease, autosomal dominant Parkinson disease, familial, type 1 (PARK1), Parkinson disease 3, autosomal dominant Lewy body (PARK3), Parkinson disease 4, autosomal dominant Lewy body (PARK4), Parkinson disease 5 (PARK5), Parkinson disease 6, autosomal recessive early-onset (PARK6), Parkinson disease 2, autosomal recessive juvenile (PARK2), Parkinson disease 7, autosomal recessive early-onset (PARK7), Parkinson disease 8 (PARK8), Parkinson disease 9 (PARK9), Parkinson disease 10 (PARK10), Parkinson disease 11 (PARK11), Parkinson disease 12 (PARK12), Parkinson disease 13 (PARK13), or mitochondrial Parkinson's disease. In some embodiments, the neuronal disease is Alzheimer's disease, Parkinson's disease, Parkinson's dementia, autism, stroke, post-traumatic stress disorder (PTSD), traumatic brain disorder (TBD), chronic traumatic encephalopathy (CTE), schizophrenia, dementia (e.g., general dementia), attention-deficit/hyperactivity disorder (ADHD), amyotrophic lateral sclerosis (ALS), frontotemporal lobar degeneration (FTLD) (e.g., FTLD-tau, FTLD-TDP, or FTLD-FUS), memory loss (e.g., age-related memory loss), hypertensive encephalopathy, or chronic stress.

In some embodiments, the neuronal disease is Alzheimer's disease (AD). Alzheimer's disease is characterized by symptoms of memory loss in the early stages of the disease. Apoε4 carriers are at greater risk of developing AD. APOε4 is believed to be less efficient than other isoforms at clearing Ac, and thus may be correlated with greater amyloid burden, tau phosphorylation, synaptotoxicity, and reduced synaptic density. Having experienced a traumatic brain injury (TBI) is another risk factor for developing AD, and studies indicate that those who experience a TBI have a significantly increased risk of AD. Cognitive decline has been correlated with the progressive loss of synapses. As the disease advances, symptoms include confusion, long-term memory loss, paraphasia, loss of vocabulary, aggression, irritability and/or mood swings. In more advanced stages of the disease, there is loss of bodily functions. Patients with Alzheimer's Disease (AD) demonstrate many characteristic neuropathies such as increased oxidative stress, mitochondrial dysfunction, synaptic dysfunction, disruption of calcium homeostasis, deposition of senile plaques and neurofibrillary tangles, and atrophy of the brain. AD related disorders include senile dementia of AD type (SDAT), frontotemporal dementia (FTD), vascular dementia, mild cognitive impairment (MCI) and age-associated memory impairment (AAMI). In some embodiments, a method of treating or preventing Alzheimer's disease is provided, comprising administering to a patient in need thereof a therapeutically effective amount of imipramine or a pharmaceutically acceptable salt thereof. In some embodiments, the patient is an Apoε2 or Apoε3 carrier. In some embodiments, the patient has suffered a TBI. In some embodiments, the patient is an Apoε4 carrier. In some embodiments, the patient is an Apoε4 carrier who has suffered a TBI.

In some embodiments, the method of treating or preventing Alzheimer's disease comprises administering to a patient in need thereof, such as those described in embodiments above, a therapeutically effective amount of semapimod, or a pharmaceutically acceptable salt thereof. In some embodiment, the method of treating or preventing Alzheimer's disease comprises administering to a patient in need thereof a therapeutically effective amount of imipramine, or a pharmaceutically acceptable salt thereof.

In some embodiments, the condition is an autism spectrum condition. Conditions on the autism spectrum are frequently associated with spine density changes that could be treated with a compound that interacts with or inhibits fascin. In some embodiments, the neuronal disease is autism. As known in the art, autism is a disorder of neural development. Without wishing to be bound by any theory, it is believed that autism, and autism spectrum conditions, affect information processing in the brain by altering how nerves and synapses connect and organize.

In further embodiments, the compositions and methods are provided for alleviating, reducing, or reversing a symptom of a neuronal disease or disorder. The symptom may be any symptom described herein.

The term “memory” and the like refer, in the usual and customary sense, to the processes by which information is encoded, stored and retrieved by a subject. The terms “encode,” “register” and the like in the context of memory refer, in the usual and customary sense, to receiving, processing and combining information impinging on the senses as chemical or physical stimuli. The term “stored” and the like in this context refer, in the usual and customary sense, to the creation of a record of the encoded information. The terms “retrieve,” “recall” and the like in this context refer, in the usual and customary sense, to calling back the stored information. Retrieval can be in response to a cue, as known in the art. In some embodiments, memory loss refers to a diminished ability to encode, store, or retrieve information. In some embodiments, the memory may be recognition memory or recall memory. In this context, “recognition memory” refers to recollection of a previously encountered stimulus. The stimulus can be e.g., a word, a scene, a sound, a smell or the like, as known in the art. A broader class of memory is “recall memory” which entails retrieval of previously learned information, e.g., a series of actions, list of words or number, or the like, which a subject has encountered previously. The method can be used to treat memory disorders that occur within the main categories of declarative and non-declarative memory, including episodic memory and “motor memory.” Methods for assessing the level of memory encoding, storage and retrieval demonstrated by a subject are well known in the art, including methods disclosed herein. For example, in some embodiments the method improves memory in a subject in need thereof, wherein the subject has a neuronal disease. In some embodiments, the method improves memory in the subject. In some embodiments, the method treats neuronal or cognitive impairment in the subject. In some embodiments, the method treats neuronal impairment in the subject. In some embodiments, the method treats cognitive impairment in the subject.

Further to any aspect disclosed herein, in some embodiments the subject suffers from brain injury. Types of brain injury include brain damage (i.e., destruction or degeneration of brain cells), traumatic brain injury (i.e., damage accruing as the result of an external force to the brain), stroke (i.e., a vascular incident which temporarily or permanently damages the brain, e.g., via anoxia), and acquired brain injury (i.e., brain damage not present at birth). In some embodiments, the method improves memory in the subject. In some embodiments, the method improves learning in the subject. In some embodiments, the method treats neuronal or cognitive impairment in the subject. In some embodiments, the method treats neuronal impairment in the subject. In some embodiments, the method treats cognitive impairment in the subject.

In some embodiments, a method for promoting spinogenesis in a patient in need thereof is provided, comprising administering to the patient a compound that interacts with or inhibits fascin. In some embodiments, a method of treating or preventing a neuronal disease or disorder is provided, comprising administering to a patient in need thereof a therapeutically effective amount of imipramine or a pharmaceutically acceptable salt thereof. In some embodiments, a compound for use in the treatment of a neuronal disease or disorder is provided, wherein the compound is imipramine or a pharmaceutically acceptable salt thereof. In some embodiments, a compound for use in the manufacture of a medicament for the treatment of a neuronal disease or disorder is provided, wherein the compound is imipramine or a pharmaceutically acceptable salt thereof. In some embodiments, the neuronal disease or disorder is selected from Alzheimer's disease, Parkinson's disease, Parkinson's dementia, autism, fragile X syndrome, and traumatic brain injury. In some embodiments, the neuronal disease or disorder is Alzheimer's disease. In some embodiments, imipramine or a pharmaceutically acceptable salt thereof, interacts with or inhibits cross-linking of f-actin. In some embodiments, imipramine or a pharmaceutically acceptable salt thereof, is anti-metastatic.

In some embodiments, a method of treating or preventing a neuronal disease or disorder is provided, comprising administering to a patient in need thereof a therapeutically effective amount of semapimod, or a pharmaceutically acceptable salt thereof, or brilacidin, or a pharmaceutically acceptable salt thereof. In some embodiments, a compound for use in the treatment of a neuronal disease or disorder is provided, wherein the compound is semapimod, or a pharmaceutically acceptable salt thereof, or brilacidin, or a pharmaceutically acceptable salt thereof. In some embodiments, a compound for use in the manufacture of a medicament for the treatment of a neuronal disease or disorder is provided, wherein the compound is semapimod, or a pharmaceutically acceptable salt thereof, or brilacidin, or a pharmaceutically acceptable salt thereof. In some embodiments, the neuronal disease or disorder is selected from Alzheimer's disease, Parkinson's disease, Parkinson's dementia, autism, fragile X syndrome, and traumatic brain injury. In some embodiments, the neuronal disease or disorder is Alzheimer's disease. In some embodiments, semapimod, or a pharmaceutically acceptable salt thereof, or brilacidin, or a pharmaceutically acceptable salt thereof, interacts with or inhibits cross-linking of f-actin. In some embodiments, semapimod, or a pharmaceutically acceptable salt thereof, or brilacidin, or a pharmaceutically acceptable salt thereof, is anti-metastatic.

### Combination Therapies

In one embodiment, the compounds disclosed herein may be used in combination with one or more additional therapeutic agent that are being used and/or developed to treat a neuronal disease or disorder.

When used for the treatment or prevention of the diseases and disorders described above, imipramine or a pharmaceutically acceptable salt thereof, may be administered together with one or more additional therapeutic agents, for example additional therapeutic agents approved for use in the treatment or prevention of the particular disease or disorder, and more particularly agents considered to form the current standard of care. Where combination therapy is envisaged, the active agents may be administered simultaneously, separately or sequentially in one or more pharmaceutical compositions.

Likewise, semapimod, or a pharmaceutically acceptable salt thereof, or brilacidin, or a pharmaceutically acceptable salt thereof, may be administered together with one or more additional therapeutic agents, such as those described herein including embodiments.

Recent strategies for the treatment of AD, therefore, include controlling the production or the aggregation state of specific isoforms of Aβ peptides. Additional strategies include preventing, reducing or removing toxic forms of phosphorylated tau. Other strategies involve small molecule targeting of enzymes that play a role in production of Aβ peptides through processing of amyloid precursor protein in an attempt to lower the abundance of Aβ peptides in the brain. Additionally, there is accruing information on the role of non-amyloid neuropathies such as tauopathy or sporadic inheritance of specific mutations in the apolipoprotein E gene, which is stimulating additional strategies to combat neurodegeneration.

### Kits

Provided herein are also kits that include compounds described herein, or a pharmaceutically acceptable salt thereof, optionally a second active ingredient, and suitable packaging. In one embodiment, a kit further includes instructions for use. In one aspect, a kit includes a compound, or a pharmaceutically acceptable salt thereof, and a label and/or instructions for use of the pharmaceutical composition in the treatment of the indications, including the diseases or conditions, described herein.

Provided herein are also articles of manufacture that include a compound described herein, or a pharmaceutically acceptable salt thereof in a suitable container. The container may be a vial, jar, ampoule, preloaded syringe, nebulizer, aerosol dispensing device, dropper, or intravenous bag.

### Pharmaceutical Compositions and Modes of Administration

Compounds provided herein are usually administered in the form of pharmaceutical compositions. Thus, provided herein are also pharmaceutical compositions that contain one or more of the compounds described herein, including generally a compound described herein, or a pharmaceutically acceptable salt thereof and one or more pharmaceutically acceptable vehicles selected from carriers, adjuvants and excipients. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The pharmaceutical compositions may be administered in either single or multiple doses. The pharmaceutical composition may be administered by various methods including, for example, rectal, buccal, intranasal and transdermal routes. In certain some embodiments, the pharmaceutical composition may be administered by intra-arterial injection, intravenously, intraperitoneally (“i.p.”), parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compositions described herein. Administration may be via, for example, capsule or enteric coated tablets. In making the pharmaceutical compositions that include at least one compound described herein or a pharmaceutically acceptable salt thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The pharmaceutical composition and any container in which it is distributed may be sterilized. The pharmaceutical composition may also contain adjuvants such as preservatives, stabilizers, emulsifiers or suspending agents, wetting agents, salts for varying the osmotic pressure, viscosity alerting agents, or buffers.

The compositions that include at least one compound described herein, such as a compound described herein, or a pharmaceutically acceptable salt thereof can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or a pharmaceutically acceptable salt thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The pharmaceutical composition may be formulated for nasal administration. Such pharmaceutical compositions may include one or more active ingredients, such as a compound described herein, or a pharmaceutically acceptable salt thereof, in varying physical states. For example, the active ingredients may be dissolved or suspended in a liquid carrier. The active ingredients may be in a dry form. The dry form may be a powder. Active ingredients in a powder may be amorphous or crystalline. For example, a compound described herein, or a pharmaceutically acceptable salt thereof, may be amorphous or crystalline. The crystalline active material may be a hydrate or a solvate.

Solid compounds, or a salt or crystal thereof, may be present in a formulation in a selected average particle size. The particles may have an average particle size (in longest dimension) of 10 nm, 100 nm, 300 nm, 500 nm, 1 μm, 10 μm, 50 μm, 100 μm, 300 μm, or 500 μm, or a range between any two values.

Administration may be by inhalation or insufflation. Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described herein. In some embodiments, the compositions are administered by the oral or nasal respiratory route. Effects may be local or systemic. In a particular embodiment, the effect is local to cranial tissues. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner. A pharmaceutical composition for inhalation or insufflation may be an aerosol.

The pharmaceutical composition may comprise a liquid suspension or solution comprising about 0.05%, about 0.1%, about 0.3%, about 0.5%, about 0.7%, about 1%, about 2%, about 3%, about 4%, or about 5% w/w of active ingredients. The liquid may comprise water and/or an alcohol. The liquid may include a pH adjusting agent such that the pH is about 1, about 2, about 3, about 4, about 5, about 6, about 7, about 8, about 9, or about 10, or a range of values therebetween.

The pharmaceutical composition may comprise a pharmaceutically acceptable preservative. Preservatives suitable for use herein include, but are not limited to, those that protect the solution from contamination with pathogenic particles, including phenylethyl alcohol, benzalkonium chloride, benzoic acid, or benzoates such as sodium benzoate. In certain some embodiments, the pharmaceutical composition comprises from about 0.01% to about 1.0% w/w of benzalkonium chloride, or from about 0.01% and about 1% v/w phenylethyl alcohol. Preserving agents may also be present in an amount from about 0.01% to about 1%, preferably about 0.002% to about 0.02% by total weight or volume of the composition.

The pharmaceutical composition may also comprise from about 0.01% to about 90%, or about 0.01% to about 50%, or about 0.01% to about 25%, or about 0.01% to about 10%, or about 0.01% to about 1% w/w of one or more of an emulsifing agent, a wetting agent or a suspending agent. Such agents for use herein include, but are not limited to, polyoxyethylene sorbitan fatty esters or polysorbates, including, but not limited to, polyethylene sorbitan monooleate (Polysorbate 80), polysorbate 20 (polyoxyethylene (20) sorbitan monolaurate), polysorbate 65 (polyoxyethylene (20) sorbitan tristearate), polyoxyethylene (20) sorbitan mono-oleate, polyoxyethylene (20) sorbitan monopalmitate, polyoxyethylene (20) sorbitan monostearate; lecithins; alginic acid; sodium alginate; potassium alginate; ammonium alginate; calcium alginate; propane-1,2-diol alginate; agar; carrageenan; locust bean gum; guar gum; tragacanth; acacia; xanthan gum; karaya gum; pectin; amidated pectin; ammonium phosphatides; microcrystalline cellulose; methyl cellulose; hydroxypropylcellulose; hydroxypropylmethylcellulose; ethylmethylcellulose; carboxymethylcellulose; sodium, potassium and calcium salts of fatty acids; mono-and di-glycerides of fatty acids; acetic acid esters of mono- and di-glycerides of fatty acids; lactic acid esters of mono-and di-glycerides of fatty acids; citric acid esters of mono-and di-glycerides of fatty acids; tartaric acid esters of mono-and di-glycerides of fatty acids; mono-and diacetyltartaric acid esters of mono-and di-glycerides of fatty acids; mixed acetic and tartaric acid esters of mono-and di-glycerides of fatty acids; sucrose esters of fatty acids; sucroglycerides; polyglycerol esters of fatty acids; polyglycerol esters of polycondensed fatty acids of castor oil; propane-1,2-diol esters of fatty acids; sodium stearoyl-2lactylate; calcium stearoyl-2-lactylate; stearoyl tartrate; sorbitan monostearate; sorbitan tristearate; sorbitan monolaurate; sorbitan monooleate; sorbitan monopalmitate; extract of quillaia; polyglycerol esters of dimerised fatty acids of soya bean oil; oxidatively polymerised soya bean oil; and pectin extract.

In a further embodiment, the pharmaceutical composition for nasal administration may be provided in the form of a powder. For example, a powdery nasal composition can be directly used as a powder for a unit dosage form. If desired, the powder can be filled in capsules such as hard gelatine capsules. The contents of the capsule or single dose device may be administered using e.g. an insufflator.

Thus, a method for treating a neuronal disorder may include the step of administering nasally a pharmaceutical composition comprising a compound described herein, or a salt thereof, to a subject in need thereof.

### Dosing

The specific dose level of an active ingredient of the present application, for example a compound described herein, of a salt thereof, for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 0.01 mg/kg may be appropriate. In some embodiments, about 0.07 and 0.03 mg/kg may be appropriate. In other embodiments a dosage of between 0.06 and 0.04 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The daily dosage may also be described as a total amount of a compound described herein administered per dose or per day. Daily dosage of a compound described herein, or a salt thereof, may be between about 1 mg and 4,000 mg, between about 2,000 to 4,000 mg/day, between about 1 to 2,000 mg/day, between about 1 to 1,000 mg/day, between about 10 to 500 mg/day, between about 20 to 500 mg/day, between about 50 to 300 mg/day, between about 75 to 200 mg/day, or between about 15 to 150 mg/day.

When administered nasally, the total daily dosage for a human subject may be between 1 mg and 1,000 mg, between about 1,000-2,000 mg/day, between about 10-500 mg/day, between about 50-300 mg/day, between about 75-200 mg/day, or between about 100-150 mg/day. In various embodiments, the daily dosage is about 10 mg, about 30 mg, about 50 mg, about 75 mg, about 100 mg, about 200 mg, about 300 mg, about 400 mg, about 500 mg, about 600 mg, about 700 mg, about 800 mg, about 900 mg, or about 1000 mg, or a range of values therebetween.

The active ingredients of the present application or the pharmaceutical compositions thereof may be administered once, twice, three, or four times daily, using any suitable mode described above. Also, administration or treatment may be continued for a number of days; for example, commonly treatment would continue for at least 7 days, 14 days, or 28 days, for one cycle of treatment. Treatment cycles are well known, and are frequently alternated with resting periods of about 1 to 28 days, commonly about 7 days or about 14 days, between cycles. The treatment cycles, in other embodiments, may also be continuous. Administration or treatment may be continued indefinitely.

In a particular embodiment, the method comprises administering to the subject an initial daily dose of about 1 to 800 mg of a compound described herein and increasing the dose by increments until clinical efficacy is achieved. Increments of about 5, 10, 25, 50, or 100 mg can be used to increase the dose. The dosage can be increased daily, every other day, twice per week, or once per week.

### EXAMPLES

The following example is included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

FIG. 1 demonstrates an analysis of synapses densities in neurons following treatments with three compounds described herein, namely, imipramine, semapimod, and brilacidin. Specifically, primary mouse hippocampal neurons were treated on day 14 in culture with one of the four compounds, each at concentrations of 10 μM, 500 nM, and 50 nM, or with control (vehicle (0.1% DMSO)) for 24 hours. Following the treatments, neurons were fixed in 4% paraformaldehyde and processed for staining with phalloidin and immunolabeling with antibodies to the postsynaptic marker PSD95, and the presynaptic marker VGlut. Images were acquired using a laser scanning confocal microscope and analyzed for the number of colocalized pre and post synaptic elements per length of dendrite. Studies were performed across 3 biological preparations of primary neurons, with a minimum of 12 dendritic segments analyzed per treatment/prep. Graphing and statistical analyses of synaptic density data were performed with Prism software.

As illustrated in FIG. 1, each of the four compounds analyzed demonstrated efficacy in promoting spinogenesis. Moreover, the binding activity are concentration dependent. For example, imipramine, semapimod, and brilacidin each demonstrated higher activities at the concentration of 10 μM than at lower concentrations.

Of the three compounds tested, imipramine is known to interact with or bind fascin at actin-binding site 2 and to interact or inhibit fascin's actin-bundling activity. It is therefore contemplated that other compounds that interact with or bind to the actin-binding site 2 would similarly have efficacy in promoting spinogenesis. Moreover, semapimod and brilacidin were determined through in silico docking analyses to have favorable docking scores to actin-binding site 3 of fascin. It is therefore contemplated that other compounds that bind to the actin-binding site 3, such as those site 3-binding compounds described herein, would also have efficacy in promoting spinogenesis. Additionally, as binding to both actin-binding sites 2 and 3 lead to efficacy, it is further contemplated that compounds that bind to fascin on other binding sites, such as actin-binding site 1 (e.g. those site 1-binding compounds disclosed herein), would also have efficacy in promoting spinogenesis.

Unless otherwise defined, all technical and scientific terms used herein have the same meaning as commonly understood by one of ordinary skill in the art to which this disclosure belongs.

The disclosures illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible.

Thus, it should be understood that although the present disclosure has been specifically disclosed by preferred embodiments and optional features, modification, improvement and variation of the disclosures embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

All publications, patent applications, patents, and other references mentioned herein are expressly incorporated by reference in their entirety, to the same extent as if each were incorporated by reference individually. In case of conflict, the present specification, including definitions, will control.

What is claimed is:

1. A method of promoting spinogenesis in a neuron, comprising contacting the neuron with imipramine, or a pharmaceutically acceptable salt thereof.

2. A method of treating or preventing a neuronal disease or disorder comprising administering to a patient in need thereof a therapeutically effective amount of imipramine, or a pharmaceutically acceptable salt thereof, provided that the neuronal disease or disorder is not a mood disorder.

3. The method of claim 2, wherein the neuronal disease or disorder is selected from Alzheimer's disease, Parkinson's disease, amyotrophic lateral sclerosis (ALS), frontotemporal dementia (FTD), and traumatic brain injury.

4. The method of claim 2, wherein the neuronal disease or disorder is Alzheimer's disease.

5. A method of promoting spinogenesis in a neuron, comprising contacting the neuron with semapimod or a pharmaceutically acceptable salt thereof.

6. A method of promoting spinogenesis in a neuron, comprising contacting the neuron with brilacidin or a pharmaceutically acceptable salt thereof.

7. A method of treating or preventing a neuronal disease or disorder comprising administering to a patient in need thereof a therapeutically effective amount of semapimod, or a pharmaceutically acceptable salt thereof.

8. The method of claim 7, wherein the neuronal disease or disorder is selected from Alzheimer's disease, Parkinson's disease, amyotrophic lateral sclerosis (ALS), frontotemporal dementia (FTD), and traumatic brain injury.

9. The method of claim 7, wherein the neuronal disease or disorder is Alzheimer's disease.

10. A method of treating or preventing a neuronal disease or disorder comprising administering to a patient in need thereof a therapeutically effective amount of brilacidin, or a pharmaceutically acceptable salt thereof.

11. The method of claim 10, wherein the neuronal disease or disorder is selected from Alzheimer's disease, Parkinson's disease, amyotrophic lateral sclerosis (ALS), frontotemporal dementia (FTD), and traumatic brain injury.

12. The method of claim 10, wherein the neuronal disease or disorder is Alzheimer's disease.
